(19) United States
(12) Patent Application Publication
(10) Pub. No.: US 2009/0054754 A1
(43) Pub. Date: Feb. 26, 2009

(54) CLINICIAN-CONTROLLED SEMI-AUTOMATED MEDICATION MANAGEMENT

(76) Inventors: Dave McMahon, Solana Beach, CA (US); Mark Ries Robinson, Albuquerque, NM (US); Mike Borrello, Carlsbad, CA (US); Richard Thompson, Dana Point, CA (US); Shonn Hendee, Carlsbad, CA (US)

Correspondence Address: V. Gerald Grafe, esq., P.O. Box 2689, Corrales, NM, 87048, US

(21) Appl. No.: 12/188,205

(22) Filed: Aug. 8, 2008

Related U.S. Application Data
(63) Continuation-in-part of application No. 11/842,624, filed on Aug. 21, 2007.

(51) Int. Cl. A61B 5/145; A61M 5/00

(52) U.S. Cl. 600/365; 604/66

(57) ABSTRACT

The present invention is directed to methods and apparatuses of medication management based upon active authorization of medication infusion by a clinician that can provide for effective management of an analyte in a patient's blood, reducing the opportunities for human error common with current manual systems while still placing final control of the medication management with the human clinician. For example, a semi-automated glucose management system can measure the glucose level in a patient's blood, recommend infusion parameters to a clinician who can authorize an infusion of glucose or insulin, and infuse the glucose or insulin into the patient.

### CROSS-REFERENCE TO RELATED APPLICATIONS

This application is a continuation-in-part of U.S. patent application Ser. No. 11/842,624, filed Aug. 21, 2007, which is incorporated herein by reference.

### FIELD OF THE INVENTION

This invention relates to the management of patient medication, and more specifically to the use of automated glucose measurement systems, glucose and insulin dosing methods, and automated infusion, without losing clinician control over the patient's care.

### BACKGROUND OF THE INVENTION

Many peer-reviewed publications have demonstrated that tight control of blood glucose (BG) significantly improves critical care patient outcomes. In particular, tight glycemic control (TGC) has been shown to reduce overall intensive care unit (ICU) mortality by 40% with significant reductions in ICU length of stay. See Van den Berghe et al., NEJM 2001; 345:1359, which is incorporated herein by reference. Historically, caregivers have treated hyperglycemia (high blood glucose) only when glucose levels exceeded 220 mg/dl. Based upon recent clinical findings, however, experts now recommend intravenous (IV) insulin administration to control blood glucose to within the normoglycemic range (80-110 mg/dl). Adherence to such strict glucose control regimens requires frequent monitoring of blood glucose and frequent adjustment of insulin infusion to achieve normoglycemia while avoiding risk of hypoglycemia (low blood glucose). In response to the demonstrated clinical benefit, internal research has revealed that approximately 82% of US hospitals have adopted some form of TGC. Furthermore, internal research has revealed that 36% of hospitals already using glycemic management protocols in their ICUs plan to expand the practice to other units and 40% of hospitals that have near-term plans to adopt TGC protocols in the ICU also plan to do so in other areas of the hospital. As research continues to show the benefits of driving patient's blood glucose levels, even lower these TGC protocols have become increasingly labor-intensive and complicated. Typical protocols today call for 44 blood glucose samples taken over a patient's three-day stay in the ICU. Dr. Krinsley has shown additional reductions in mortality by maintaining blood glucose down to a level in the 80 to 90 mg/dl range. See Krinsley et al., Mayo Clin Proc, 78, 1471 (2003), which is incorporated herein by reference.

Given the compelling evidence for improved clinical outcomes associated with tight glycemic control, hospitals are under pressure to implement TGC as the standard of practice for critical care and cardiac surgery patients. Clinicians and caregivers have developed TGC protocols that use IV insulin administration to maintain normal patient glucose levels. To be safe and effective, these protocols require frequent blood glucose monitoring, manual calculation of glucose or insulin infusion parameters, and manual control of infusion systems.

To implement TGC protocols using today's manual, finger-stick technologies requires many steps, is technique sensitive and has opportunities for user errors. Using these technologies require removal of a blood sample, placement of just the right amount of blood on a test strip, evaluation of the result, determination of the correct glucose or insulin dose using a complex algorithm, and finally adjustment to the insulin infusion rate. In a recent study published in the America College of Surgeons in 2006, Taylor et al. noted that while implementing a TGC protocol, errors were found in the implementation of the protocol in 47% of all patients. Half of the errors were considered major, such as missing two or more glucose measurements in a row and insulin dosing errors. See Taylor et al., Journal of American College of Surgeons, 202, 1 (2006), which is incorporated herein by reference. The current manual method of TGC requires multiple types of equipment and at least two hours of nursing time per patient per day to implement. Even with all of this equipment and time spent, the targeted glycemic range of 80-110 mg/dl is difficult to achieve and maintaining patients in this range is even more difficult.

Medication errors are a significant and growing problem that can result in tragic loss of life and significant cost increases to the health-care community. Recent studies have listed medical errors as the eighth leading cause of death, ahead of motor vehicle accidents, breast cancer or AIDS. The American Hospital Association estimates that medical errors account for between 44,000 and 98,000 U.S. deaths each year. From a financial perspective, research indicates that nationally, the annual cost of preventable adverse drug events in the U.S. is about $6 billion. Over 770,000 patients are injured because of medication errors every year. Medication errors occur in nearly 1 of every 5 doses given to patients in the typical hospital. Reported rates of adverse drug events (ADEs) range from 2.4 to 6.1 ADEs per 100 admissions or discharges, or 9.1 to 19 ADEs per 1000 patient days.

Medication errors often arise from errors in drug administration, which account for 38% of medication errors. Only 2% of drug administration errors are intercepted. Safety at the point of care is one of the greatest areas for potential improvement in the medication use process. 54% of potential ADEs are associated with IV medications. Studies have found that ADEs occur between 2.9 and 3.7 percent of hospitalizations. 61% of the serious and life-threatening errors are associated with IV medications. Insulin has been described as the most dangerous IV medicine, with special protocols and checks recommended to help prevent life-threatening errors. See “Reducing Variability in High Risk Intravenous Medication Use”, Center for Medication Safety and Clinical Improvement, 2005, Cardinal Health, which is incorporated herein by reference.

The first concepts of an artificial pancreas were conceived in the 1970's. Such systems offer the promise of complete automation—the patient's blood glucose would be completely and perfectly controlled with no human user intervention. See “Report of the Automated Control of Insulin Levels Committee”, Committee Report (DRA 5), Institute for Alternative Futures, p. 9, September, 2006, which is incorporated herein by reference. However, any error in the measurement, infusion determination, or infusion system can lead to catastrophic medication errors, and so such systems have seen little use.

Accordingly, there is a need for a semi-automated medication management system that reduces the chance of missed measurements, infusion calculation errors, or infusion control errors while still involving a human clinician in the final infusion decision.

### SUMMARY OF THE INVENTION

The present invention comprises methods and apparatuses for medication management based upon active authorization of medication infusion by a clinician that can provide for effective management of an analyte in a patient's blood, reducing the opportunities for human error common with current manual systems while still placing final control of the medication management with the human clinician. For ease of understanding, the description herein generally refers to the control of glucose levels in a patient's blood by infusion of glucose or insulin, but other analytes (i.e., substances) or medications can be substituted with appropriate system adjustments as needed. For example, the system can be used for the measurement of hematocrit to control a patient's red blood cell count, or for the infusion of the heparin for anticoagulation, or to control the levels of substances such as Dopamine or Propofol.

An embodiment of the present invention is a semi-automated glucose management system, comprising a glucose measurement system, adapted to measure the glucose level in a patient's blood, or an indicator thereof; an infusion recommendation system, adapted to recommend infusion parameters based on information comprising the measured blood glucose level; an infusion control system, adapted to infuse glucose or insulin into the patient, and means for a clinician to authorize an infusion of glucose or insulin into the patent by the infusion control system based on a recommendation of infusion parameters by the infusion recommendation system. The glucose measurement system, infusion recommendation system, and infusion control system can be integrated in a single unit. The glucose management system can further comprise means for automated record keeping for blood glucose level measurements, glucose and insulin infusion parameters, identity of the authorizing clinician, and the timing of blood glucose level measurements and infusion parameters.

A glucose measurement system can comprise means for measurement of the patient's blood glucose level at predetermined time intervals or at time intervals determined from patient or treatment parameters. The measurement system can use an optical measurement of analyte in whole blood or measurements of analyte in portions of blood samples after removal of substantially all the red blood cells in the portion. The present invention can comprise apparatuses useful for automatically determining analyte values such as blood glucose levels. Such apparatuses can comprises a fluid access system, adapted to withdraw a sample of a bodily fluid such as blood from a patient; an analyte measurement system, adapted to measure the value of an analyte such as glucose level from the blood sample; and a controller, adapted to cause the fluidics system to withdraw a fluid sample for measurement at times determined by patient conditions, environmental conditions, or a combination thereof.

The information of the infusion recommendation system can further comprise previous values of the patient's blood glucose level, the patient's previous response to previous glucose or insulin infusion, or the patient's glucose treatment characteristics. The patient's glucose treatment characteristics can comprise a target blood glucose level or a target range, a glucose measurement frequency, the patient's age, or the patient's diabetic status.

The infusion recommendation system can comprise an imbedded algorithm to recommend the infusion parameters. The clinician can vary the infusion of glucose or insulin from the recommendation of the infusion recommendation system only if a certain clinician authorization level is provided.

The present invention is further directed to a method for semi-automated medication management, comprising measuring an analyte level in a patient's blood, or an indicator thereof, with a analyte measurement system; recommending infusion parameters based on information comprising the measured blood analyte level with a medication recommendation system; authorizing an infusion of medication into the patent by an infusion control system based on the recommendation of infusion parameters by the infusion recommendation system; and infusing medication into the patient with the infusion control system following the authorizing by a clinician. For example, the method can comprise measuring the blood glucose level, recommending infusion parameters, authorizing an infusion of glucose or insulin by the clinician, and infusing the glucose or insulin into the patient.

Advantages and novel features of the present invention will become apparent to those skilled in the art upon examination of the following description or can be learned by practice of the invention. The advantages of the invention can be realized and attained by means of the methods, example embodiments, and combinations specifically described in the disclosure and in the appended claims.

### BRIEF DESCRIPTION OF THE DRAWINGS

The accompanying drawings, which are incorporated in and form part of the specification, illustrate the present invention and, together with the description, describe the invention. In the drawings, like elements are referred to by like numbers.

FIG. 1 is a schematic illustration of a semi-automated glucose management system comprising separate glucose measurement, infusion recommendation, and infusion control systems.

FIG. 2 is a schematic illustration of a semi-automated glucose management system comprising integrated glucose measurement, infusion recommendation, and infusion control systems.

FIG. 3 is a schematic illustration of a semi-automated glucose management system comprising integrated glucose measurement, infusion recommendation, and infusion control systems.

### DETAILED DESCRIPTION OF THE INVENTION

The present invention comprises methods and apparatuses of medication management based upon active authorization of medication infusion by a clinician that can provide for effective management of an analyte in a patient's blood, reducing the opportunities for human error common with current manual systems while still placing final control of the medication management with the human clinician.

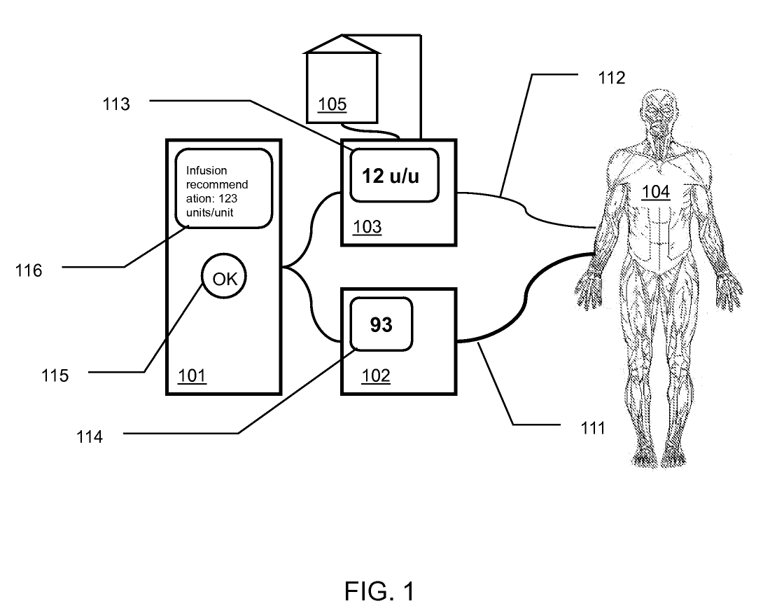

FIG. 1 is a schematic illustration of an exemplary glucose management system according to the present invention. An automated glucose measurement system 102 is in communication 111 with a patient 104 such that the automated glucose measurement system can determine the glucose level, or concentration value, of the patient's blood, or an indicator thereof. A variety of glucose measurement systems can be used with the present invention as described later. The automated glucose measurement system can communicate the present or most recent glucose level, or other information relating to the patient's condition, e.g., by a visible display 114 or by audible signals such as warning tones or synthesized or recorded speech. Information concerning the patient's blood glucose is communicated from the automated glucose measurement system 102 to an infusion recommendation system 101. The information can comprise the present or most recent value of the patient's blood glucose, and can also comprise information such as several historical values of the patient's blood glucose, and can also comprise time intervals or measurement times of such values.

The infusion recommendation system 101 can comprise, as an example, an automated data processor or computer programmed to determine recommended infusion, or dosing, parameters. The determination can be based on the current patient glucose level, and can also be based on information such as previous values, previous response to previous infusion, glucose treatment characteristics, or any other patient information that might be available (e.g., target glucose level or acceptable glucose target range, acceptable measurement frequency, patient age, diabetic or other medical status, patient caloric input, or other therapies, such as vasopressors, steroids or heparin, that can affect metabolic action or patient response). The system 101 can determine a single infusion recommendation, or a range of recommendations, or a plurality of recommendations based on various parameters (e.g., one recommendation if an abrupt change in glucose is desired or another recommendation if a more gradual change is desired). The clinician can change parameters of the infusion recommendation algorithm, or alternatively can change the parameters only if a certain clinician authorization level is provided (to avoid treatment regime changes by unqualified personnel).

The system 101 communicates the infusion recommendation or dosing parameter recommendation to a human clinician, for example by a visible display 116 or by audible signals such as warning tones or synthesized or recorded speech. The clinician can communicate with the system to adjust the recommended infusion or dosing, or to select among several recommendations, and finally to authorize the infusion, for example by pressing a button 115, by voice commands, by interaction with a touch-sensitive screen, or by other signal communicated to the system. As another example, communication with the clinician can be accomplished remotely, for example by phone or computer network with remote terminals, cell phones, or personal digital assistants (PDAs). As further examples, communication with the clinician can be to a monitoring station such as a central nursing station, or integrated with a patient monitoring system, such as those available from GE Healthcare or Marquette Medical Systems. The present invention can be compatible with systems like those described in U.S. Pat. No. 7,398,183, which is incorporated herein by reference.

After a clinician authorizes the infusion, the infusion recommendation system 101 communicates the authorized infusion to an infusion control system 103. The communication can comprise any method that allows the infusion control system 103 to implement the authorized infusion; for example a wired data connection or a wireless data connection that communicates the full specification of the authorized infusion, a change from the previous authorized infusion, or a specification only of a readily changeable infusion parameter, such as flow rate. The infusion control system 103 can, for example, draw fluid such as insulin from an attached bag 105 for transfer 112 to the patient 104, and can communicate information concerning the ongoing infusion to the clinician, for example, using a visible display 113. After some interval, for example a predetermined interval for communication with the clinician, or some other interval such as those described below, the automated glucose measurement system 102 can again determine the patient's blood glucose and the recommendation process repeated. Suitable infusion control systems can comprise intravenous infusion systems, such as commercially available IV pumps, subcutaneous infusion systems, or other methods of delivery (e.g., insulin or glucose injections, patches, pills, etc.) responsive to the infusion recommendation system (e.g., by instructing a clinician as to a dose, or controlling a medication delivery system such as Pyxis® (a trademark of Cardinal Health Inc)). A confirmation from a clinician that the infusion was completed can also be accepted as an additional safety and verification step.

The glucose management system can also provide for automated record keeping. For example, an electronic or paper log can be created, with information such as glucose measurements, infusion parameters, infusion recommendations, identity of the authorizing clinician, and times of various events. The system can also perform infusion recommendations using a remote or central computer or server, and then communicate with the clinician using a measurement system interface or an infusion system interface. The system can also perform medication verification or safety checks, for example by verifying that infusion recommendations are within applicable guidelines for infusion as stored in a hospital patient management system or as established by a hospital pharmacy control system or medication delivery system (such as the Guardrails® Safety Software system or the Pyxis® system from Cardinal Health). The system can also accommodate automated medication ordering responsive to the known and recommended infusion or dosing parameters. The system can also use automated tracking of medications, such as bar code scanning, to verify that appropriate medications (e.g., type, composition, concentration) are used with appropriate patient (e.g., patient-specific composition).

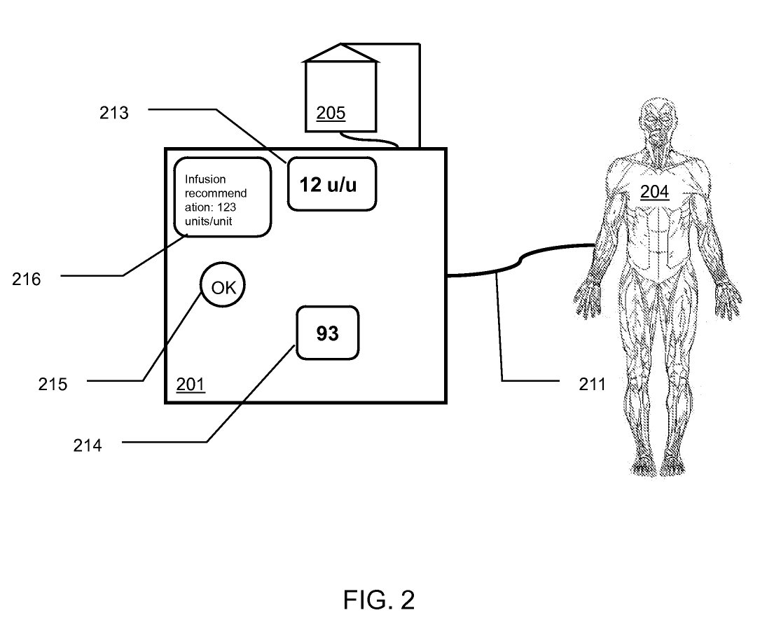

FIG. 2 is a schematic illustration of another exemplary glucose management system according to the present invention. An integrated system 201 is in communication with a patient 204 such that the system 201 can determine the patient's blood glucose level and can infuse one or more therapeutic agents 205, such as insulin or glucose, into the patient 204 after authorization from the clinician by an authorization means 215. The system 201 is an integrated unit that provides the operations for the automated glucose measurement system 102, the infusion recommendation system 101, and the infusion control system 103 described in relation to FIG. 1.

The integrated system 201 can provide some advantages over a system of discrete subsystems. For example, communication links can be internal to the integrated system, avoiding possible problems from unplugged cords or the like. Also, although FIG. 2 shows multiple display screens 213, 216, 214, an integrated system can use a single display for relevant communications. Also, an integrated system can, in some applications, make use of a single access into the patient, and hence use a single sterile tubing set and pumps. Also, an integrated system can more easily make use of more complex infusion recommendation algorithms, since the integrated system can access comprehensive information related to glucose management, such as measurement values and history, infusion history, treatment guidelines, infusion performance, etc.

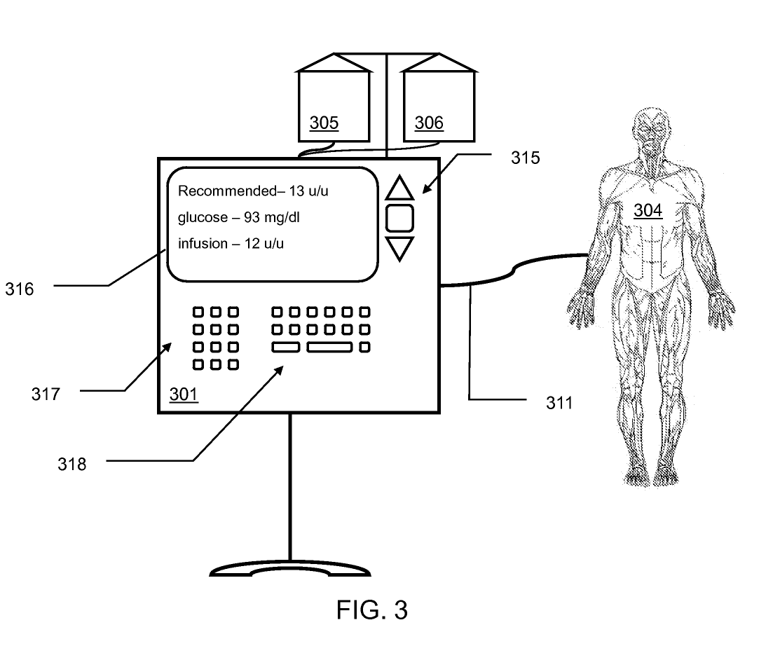

FIG. 3 is a schematic illustration of another exemplary glucose management system according to the present invention. An integrated system 301 is in communication with a patient 304 such that the system 301 can determine the patient's blood glucose level and can infuse one or more therapeutic agents 305 and 306, such as insulin or glucose, into the patient 304. The integrated system 301 is adapted to supply the operations described for the automated glucose measurement system 102, the infusion recommendation system 101, and the infusion control system 103 described in relation to FIG. 1. The integrated system 301 can communicate with a clinician via a single display 316, on which information concerning the recommended infusion can be displayed as well as other information related to patient or system status, for example patient glucose level, glucose trends, current infusion rate, system status such as state of insulin supply, glucose management scheme or constraints. The display can communicate via text, and can use graphical displays where more effective. The system can also communicate by audible signals such as warning of confirmation tones or synthesized or recorded speech. The clinician can control the system 301 in numerous ways, for example via a numeric keypad 317, an alphanumeric or special purpose keypad 318, increment/decrement and accept buttons 315, all shown in the figure for illustrative purposes, or via remote control input devices, speech or sound recognition, or combinations thereof.

### Automated Glucose Measurement System

A range of automated glucose measurement systems can be used with the present invention, for example those described in U.S. patent application Ser. No. 11/352,956 “Apparatus and methods for analyzing body fluid samples”, filed Feb. 13, 2006; Ser. No. 11/316,407 “Apparatus and methods for analyzing body fluid samples”, filed Dec. 21, 2005; Ser. No. 10/850,646 “Analyte determinations”, filed May 21, 2004; Ser. No. 11/679,826 “Blood Analyte Determinations”, filed Feb. 27, 2007; Ser. No. 11/679,837 “Analyte Determinations”, filed Feb. 28, 2007; Ser. No. 11/679,839 “Analyte Determinations”, filed Feb. 28, 2007; and Ser. No. 11/679,835 “Analyte Determinations”, filed Feb. 27, 2007; each of which is incorporated herein by reference. For example, the glucose measurement system can be adapted to withdraw a volume of blood from a patient, measure the glucose concentration in the blood, and reinfuse at least a portion of the volume of withdrawn blood back into the patient.

The automated glucose measurement system can collect measurements at predetermined intervals, for example at intervals established by direct input from the clinician. Alternatively, the automated glucose measurement system can collect measurements at intervals determined from patient or treatment parameters. For example, a specific management protocol can be communicated to the glucose measurement system, and the protocol can include specification of glucose measurement intervals. Such intervals can be constant for a particular protocol, and can vary based on parameters such as the most recent, or several most recent, glucose values. Examples of suitable control of glucose measurement times are disclosed in U.S. patent application Ser. No. 11/842,624, “Variable Sampling Interval for Blood Analyte Determination,” which is incorporated herein by reference. The present invention can allow additional information to be included in the sampling time determination. For example, the infusion rate can be used to help determine the sampling interval, and the patient's previous response to previous infusion conditions can be used to make a patient-specific physiological model more accurate.

The patient's blood glucose level can be measured at time intervals determined by determining whether a change in a patient condition, a change in an environmental condition, or a combination thereof, indicates a measurement should be made. This determination can comprise applying a physiologic model to the patient's condition, the environmental condition, or a combination thereof, and, if the physiological model indicates a blood glucose level that approaches a threshold value, then indicating that a measurement should be made. The physiologic model can comprise a model based on the interactions illustrated in the Netter diagram, an AIDA blood glucose simulator model, a Chase model, a Bergman model, a compartment model with differential equations, an insulin pharmacokinetics and distribution model, a glucose pharmacokinetics and distribution model, a meal model, a glucose/insulin pharmacodynamic model, an insulin secretion and kinetics model, or a combination of two or more of the preceding models.

### Infusion Recommendation System

The insulin or glucose dose can be recommended by an imbedded algorithm in the infusion recommendation system. Initially the algorithm can utilize patient data, such as starting blood glucose, height, weight, etc. to calculate an initial insulin or glucose dose. As additional blood glucose measurements are taken, the algorithm can “learn” how a specific patient metabolizes insulin and glucose and adjust accordingly to recommend the proper insulin or glucose dose to achieve the desired glucose range. Additionally, the algorithm can contain adjustable parameters the clinician can utilize to adjust the level of aggressiveness in the patient's treatment. Additional physiological patient changes and or medications can also factor into insulin and glucose recommendations, such as the use of vasopressors in a patient may influence their ability to metabolize insulin.

The insulin recommendation can be achieved, for example, utilizing the algorithms described in U.S. patent application Ser. No. 11/131,707 to Flanders and U.S. patent application Ser. No. 11/529,224 to Grounsel, each of which is incorporated herein the reference. As described by Flanders, an algorithm to calculate the proper amount of insulin to be delivered to the patient can be a known algorithm, such as the Protocol of Bode et al., or can be a proprietary algorithm or some other proven calculation. For example, Grounsel describes a means for calculating an optimum BG dosing or insulin infusion rate (DR, in units of insulin/time), according to the formula:

DR=(BG−K)×SF

where BG is the patient blood glucose level (mg/dl), K is a constant (where 40<K<80, depending on the type of BG measurement) and SF is a sensitivity factor that depends on the clinician's empirical input or protocol. The sensitivity factor is the patient's sensitivity to insulin (i.e., the speed at which the patient's blood sugar falls after an infusion of insulin). The sensitivity factor can be adjusted in many ways, including utilizing rules engines based on the rate of change of blood glucose level over time. For example, a BG change greater than 30 mg/dl can indicate an increase in the sensitivity factor by 10%. These same rules engines can drive more frequent measurements during periods of drastic change in the patients' blood glucose. Additionally, a clinician can manually increase or decrease the sensitivity factor via the user interface based on their desire to increase or decrease the amount of insulin infused. Additionally, the system can automatically use information from previous measurements and infusion rates to adjust the sensitivity.

A glucose infusion can also be based on a user configurable target and rules engine i.e., a BG reading less than 40 mg/dl can trigger a recommendation of a glucose infusion can could be given by the controlled infusion system. This can also trigger more frequent measurements due to the desire to quickly return the patient to a normal glycemic level (80-120 mg/dl).

The infusion recommendation system can further be used as a glucose measurement recommendation system. In such an embodiment, the recommendation system determines an appropriate time to measure glucose (or another substance or analyte). The recommendation system then communicates to a clinician a recommendation that a measurement be made. The clinician can then authorize the measurement, or postpone the measurement, or defer action on the recommendation. See U.S. patent application Ser. No. 11/842,624.

### Infusion Control System

The infusion control system can comprise an IV infusion pump. Intravenous infusion pumps have been used in hospitals for decades and there are many systems commercially available today. Three market leading IV infusion pump companies are Baxter Healthcare Inc.; Alaris Medical Systems, A Cardinal Health Company; and Hospira, Inc. All three of these companies provide both large volume pumps and syringe pumps. Examples of these commercially available products are: Baxter Colleague model 2M8151, Hospira model Plum A+, Alaris Medical Systems, Alaris System. Other examples include those described in U.S. Pat. No. 5,709,534, which is incorporated herein by reference.

The control of the IV infusion pump can be achieved by integrating a pumping module into an Automated Blood Glucose System (ABGM). Control of the infusion pump can be achieved via the ABGM's user interface. Additionally control of a stand-alone IV infusion pump can be achieved utilizing software commands transmitted from the ABGM via a server installed at the hospital or directly through wireless connectivity available on the IV infusion pump. Currently the Alaris Medical Systems, Alaris IV Infusion System, and the Hospira system have the capability to receive commands via a server.

The particular sizes and equipment discussed above are cited merely to illustrate particular embodiments of the invention. It is contemplated that the use of the invention can involve components having different sizes and characteristics. It is intended that the scope of the invention be defined by the claims appended hereto.

We claim:

1. A semi-automated medication management system, comprising:
an analyte measurement system, adapted to measure the level of an analyte in a patient's blood, or an indicator thereof;
an infusion recommendation system, adapted to recommend medication infusion parameters based on information comprising the measured blood analyte level;
an infusion control system, adapted to infuse a medication into the patient, and
an authorization system adapted to allow a clinician to authorize an infusion of the medication into the patent by the infusion control system based on a recommendation to the of infusion parameters by the infusion recommendation system.

2. The medication management system of claim 1, wherein the analyte comprises glucose and the medication comprises glucose or insulin.

3. The medication management system of claim 1, wherein the analyte comprises hematocrit.

4. The medication management system of claim 1, wherein the medication comprises heparin.

5. The medication management system of claim 1, wherein the analyte measurement system, infusion recommendation system, and infusion control system are integrated into a single unit.

6. The medication management system of claim 1, wherein the analyte measurement system is adapted to measure the patient's blood analyte level at predetermined time intervals.

7. The medication management system of claim 1, wherein the analyte measurement system is adapted to measure the patient's blood analyte level at time intervals determined from patient or treatment parameters.

8. The medication management system of claim 1, wherein the analyte measurement system is adapted to measure the patient's blood analyte level at time intervals determined by determining whether a change in a patient condition, a change in an environmental condition, or a combination thereof, indicates a measurement should be made.

9. The medication management system of claim 8, wherein determining whether a change in patient condition, a change in environmental condition, or a combination thereof, comprises applying a physiologic model to the patient's condition, the environmental condition, or a combination thereof, and, if the physiological model indicates a blood analyte level that approaches a threshold value, then indicating that a measurement should be made.

10. The medication management system of claim 9, wherein the physiologic model comprises: a model based on the interactions illustrated in the Netter diagram, an AIDA blood glucose simulator model, a Chase model, a Bergman model, a compartment model with differential equations, an insulin pharmacokinetics and distribution model, a glucose pharmacokinetics and distribution model, a meal model, a glucose/insulin pharmacodynamic model, an insulin secretion and kinetics model, or a combination of two or more of the preceding models.

11. The medication management system of claim 1, wherein the analyte measurement system is adapted to withdraw a volume of blood from a patient, measure the analyte concentration in the blood, and reinfuse at least a portion of the volume of withdrawn blood back into the patient.

12. The medication management system of claim 1, wherein the information of the infusion recommendation system further comprises previous values of the patient's blood analyte level, that patient's previous response to previous medication infusion, the patient's medication treatment characteristics, or a combination thereof.

13. The medication management system of claim 12, wherein the patient's medication treatment characteristics comprise a target blood analyte level or a target range, an analyte measurement frequency, the patient's age, or the patient's medical status, or a combination thereof.

14. The medication management system of claim 1, wherein the infusion recommendation system comprises an imbedded algorithm that can recommend the infusion parameters.

15. The medication management system of claim 1, wherein the infusion recommendation system further comprises an analyte measurement recommendation system to determine a recommended time to measure the analyte level in the patient's blood.

16. The medication management system of claim 1, wherein the infusion control system comprises an intravenous infusion pump or subcutaneous infusion means.

17. The medication management system of claim 1, wherein the authorization system comprises means for the clinician to vary the infusion of medication from the recommendation of the infusion recommendation system only if a certain clinician authorization level is provided.

18. The medication management system of claim 1, wherein the authorization system comprises means for the clinician to communicate remotely with the infusion recommendation system or the infusion control system.

19. The medication management system of claim 1, further comprising means for confirming that the medication infusion has been completed.

20. The medication management system of claim 1, further comprising means to perform a medication verification or safety check.

21. The infusion management system of claim 1, further comprising means for automated record keeping for at least one of: blood analyte level measurements, medication infusion infusion parameters, identity of the authorizing clinician, the timing of blood analyte level measurements, and the timing of infusion parameter adjustments.

22. A method for semi-automated medication management, comprising:
measuring an analyte level in a patient's blood, or an indicator thereof, with an analyte measurement system;
recommending infusion parameters based on information comprising the measured blood analyte level with an infusion recommendation system;
communicating with a clinician to authorize an infusion of medication into the patent by an infusion control system based on the recommendation of infusion parameters by the infusion recommendation system.
infusing medication into the patient with the infusion control system following the authorizing by the clinician.

23. The method of claim 22, wherein the analyte comprises glucose and the medication comprises glucose or insulin.

24. The medication management system of claim 1, wherein the infusion control system is adapted to infuse one or more of a plurality of medications into a patient, and wherein the infusion recommendation system is adapted to indicate which of the plurality of medications is recommended to infuse.
